(19) United States
(12) Patent Application Publication
(10) Pub. No.: US 2014/0017216 A1
(43) Pub. Date: Jan. 16, 2014

(54) PESTICIDAL MIXTURES

(71) Applicants: Clark D. Klein, Pittsboro, NC (US); Rebecca Willis, Game, NC (US); Luis Jose, Cary, NC (US); Nigel Ames, Raleigh, NC (US)

(72) Inventors: Clark D. Klein, Pittsboro, NC (US); Rebecca Willis, Game, NC (US); Luis Jose, Cary, NC (US); Nigel Ames, Raleigh, NC (US)

(73) Assignee: BAYER CROPSCIENCE LP, Research Triangle Park, NC (US)

(21) Appl. No.: 14/008,897

(22) PCT Filed: Mar. 30, 2012
(86) PCT No.: PCT/US2012/031690

§ 371 Date: Sep. 30, 2013

Related U.S. Application Data
(60) Provisional application No. 61/469,845, filed on Mar. 31, 2011.

(30) Foreign Application Priority Data
May 9, 2011 (EP) 11165269.9

(51) Int. Cl. A01N 63/00; A61K 35/74; A61K 31/277; A01N 37/34

(52) U.S. Cl. CPC A01N 63/00; A01N 37/34; A61K 35/74; A61K 31/277
USPC 424/93.43

(57) ABSTRACT

The present invention relates to a pesticidal mixture comprising, as active I) at least one active compound I selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and 2) at least one active compound II selected from the groups A.I to A.27 as defined in the description, in synergistically effective amounts

### CROSS-REFERENCE TO RELATED APPLICATIONS

This application claims the benefit or U.S. Patent Application No. 61/469,645, filed Mar. 31, 2011, in accordance with 35 U.S.C. Section 119(e) and European Patent Application No. 11 165 269.9-2103, in accordance with 35 U.S.C. Section 119(a), filed in the European Patent Office May 9, 2011. The contents of each of the above-referenced applications are incorporated herein by reference,

### FIELD OF INVENTION

The present invention relates to mixtures of active ingredients having synergistically enhanced action and to methods comprising applying said mixtures.

### BACKGROUND OF INVENTION

One typical problem arising in the field of pest control lies in the need to reduce the dosage rates of the active ingredient in order to reduce or avoid unfavorable environmental or toxicological effects whilst still allowing effective pest control.

Another problem encountered concerns the need to have available pest control agents which are effective against a broad spectrum of pests.

There also exists the need for pest control agents that combine knock-down activity with prolonged control, that is, last action with long lasting action.

Another difficulty in relation to the use of pesticides is that the repeated and exclusive application of an individual pesticidal compound leads in many cases to a rapid selection of pests which have developed natural or adapted resistance against the active compound in question. Therefore there is a need for pest control agents that help prevent or overcome resistance.

It was therefore an object of the present invention to provide pesticidal mixtures which solve at least one of the discussed problems as reducing the dosage rate, enhancing the spectrum of activity or combining knock-down activity with prolonged control or as to resistance management.

### SUMMARY OF INVENTION

We have found that this object is in part or whole achieved by the combination of active compounds defined below.

The present invention relates to a pesticidal mixture comprising, as active compounds,
1) at least one active compound I selected from the group consisting or the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and
2) at least one active compound II selected from the groups A.1 to A.27:

A.1. Organo(thio)phosphate compounds selected from the group consisting of acephate, azamethiphos, azinphos-ethyl, azinphos-methyl, chlorethoxyfos, chlorfenvinphos chlormephos, chlorpyrifos, chlorpyrifos-methyl, coumaphos, cyanophos, demeton-S-methyl, diazinan dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion.

A.2. Carbamate compounds selected from the group consisting of aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylearb and triazamate;

A.3. Pyrethroid compounds selected from the group consisting of acrinathrin, allethrin, d-cis-trans allethrin, d-trans allethrin, bifenthrin, bioallethrin, bioallethrin S-cylclopententyl, bioresmethrin, cycloprothrin, cyfluthrin, beta-cyfluthrin, cyhalothrin, lambda-cyhalothrin gamma-cyhalothrin, cypermethrin, alpha-cypermethrin, beta-cypermetbrin, theta-cypermethrin, zeta-cypermethrin, cyphenothrin deltametbrin, empenthrin, esfenvalerate, etofenprox, fenpropathrin, fenvalerate, flucythrinate, flumethrin, taufluvalinate, halfenprox, imiprothrin, metofluthrin, permethrin, phenothrin, prallethrin, profluthrim, pyrethrin (pyrethrum), resmethrin, silafluofen, telluthrin, tetramethrin, tralomethrin and transfluthrin;

A.4. Juvenile hormone mimics selected from the group consisting of hydroprene, ki-noprene, methoprene, fenoxyearb and pyriproxyfen;

A.5. Nicotinic receptor agonists/antagonists compounds selected from the group consisting of acetamiprid, bensultap, cartap hydrochloride, clothianidin, dinotefuran, imidacloprid, thiamethoxam, nitenpyrarn, nicotine, spinosad (allosteric agonist), spinetoram (allosteric agonist), thiacloprid, thiocyclam, thiosultapsodium and AKD1022.

A.6. GABA gated chloride channel antagonist compounds selected from the group consisting of chlordane, endosullan, gamma-HCH (lindane); ethiprole, fipronil, pyrafluprole and pyriprole;

A.7. Chloride channel activators selected from the group consisting of abamectin, emamectin benzoate, milbemectin and lepimectin;

A.8. METII compounds selected from the group consisting of fenazaquin, fenpyroximate, pyrimidifen, pyridaben, tebufenpyrad, tolleupyrad, flufenerim and rotenone;

A.9. METII and III compounds selected from the group consisting of acequinocyl, fluacyprim and hydramethylnon;

A.10. Uncouplers of oxidative phosphorylation selected from the group consisting of chlorfenapyr and DNOC;

A.11. Inhibitors of oxidative phosphorylation selected from the group consisting of azocyclotin, cyhexatin, diafenthiuron, fenbutatin oxide, propargite and tetradifon;

A.12. Moulting disruptors selected from the group consisting of cyromazine, chromafenozide, halofenozide, methoxyfenozide and tebufenozide;

A.13. Synergists selected from the group consisting of piperonyl butoxide and tribufos;

A.14. Sodium channel blocker compounds selected from the group consisting of indoxacarb and metaflumizone;

A.15. Fumigants selected from the group consisting of methyl bromide, chloropierin and sulfuryl fluoride;

A.16. Selective feeding blockers selected from the group consisting of crylotie, pymetrozine and flonicamid;

A.17. Mite growth inhibitors selected from the group consisting of clofentezine, hexythiazox and etoxazole;

A.18. Chitin synthesis inhibitors selected from the group consisting of buprofezin, bistrifluron, chlorfluazuron, diflubenzuron, flucycloxuron, flufenoxuron, hexaflumuron, lufenuron, novaluron, noviflumuron, teflubenzuron and triflumuron;

A.19. Lipid biosynthesis inhibitors selected from the group consisting of spirodiclofen, spiromesifen, and spirotetramat;

A.20. Octapaminergic agonsist: amitraz;

A.21. Ryanodine receptor modulators: flubendiamide and the phtalamid compound (R)-, (S)-3-Chlor-N1-{2-methyl-4-[1,2,2,2-tetrafluor-1-(trifluormethyl)ethyl]phenyl}-N2-(1-methyl-2-methylsulfonylethyl)phthalamid (A.21.1),

A.22. Isoxazoline compounds selected from the group consisting of 4-[5-(3,5-Dichloro-phenyl)-5-trifluoromethyl-4,5-dihydro-isoxazol-3-yl]-2-methyl-N-pyridin-2-ylmethyl-benzamide (A.22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (A.22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (A.22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (A.22.4) and 4-[5-(3,5-Dichlorophenyl)-5-trifluoromethyl-4,5-dihydro-isoxazol-3-yl]-N-[(methoxyimino)methyl]-2-methylbenzamide (A.22.5);

A.23. Anthranilamide compounds selected from the group consisting of chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (A.23.1), 5-Bromo-2-(3-chloro-pyridin-2-yl)-2H-1-pyrazole-3-carboxylic acid [2-chloro-4-cyano-6-(1-cyclopropyl-ethylcarbamoyl)-phenyl]-amide (A.23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (A.23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (A.23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (A.23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (A.23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (A.23.7), N′-(2-{[5-Bromo-2-(3-chloro-pyridin-2-yl)-2H-pyrazole-3-carbonyl]-amino}-5-chloro-3-methyl -benzoyl)-N′-methyl-hydrazinecarboxylic acid methyl ester (A.23.8), N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (A.23.9), N′-(3,5-Dibromo-2-{[5-bromo-2-(3-chloro-pyridin-2-yl)-2H-pyrazole-3-carbonyl]-amino}-benzoyl)-hydrazinecarhoxylic acid methyl ester (A.23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (A.23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (A.23.12);

A.24. Malononitrile compounds selected from the group consisting of 2-(2,2,3,3,4,4,5,5-octafluoropentyl)-2-(3,3,3-trifluoro-propyl)malononitrite (CF2H—CF2-CF2-CF2-CH2-C(CN)2-CH2-CH2-CF3) (A.24.1) and 2-(2,2,3,3,4,4,5,5-octafluoropentyl)-2-(3,3,4,4,4-pentafluorobutyl)-malonodinitrile (CF2H—CF2-CF2-CF2-CH2-C(CN)2-CH2-CH2-CF2-CF3) (A.24.2);

A.25. Microbial disruptors selected from the group consisting of Bacillus thuringiensis subsp. Israelensi, Bacillus sphaericus, Bacillus thuringiensis subsp. Aizawai, Bacillus thuringiensis subsp. Kurstaki, and Bacillus thuringiensis subsp. Tenebrionis;

A.26. Aminofuranone compounds selected from the group consisting of 4-{[(6-Bromopyrid-3-yl)methyl](2-fluoroethyl)amino}furan-2(5H)-on (A.26.1), 4-{[(6-Fluoropyrid-3-yl)methyl](2,2-difluoroethyl)amino}furan-2(5H)-on (A.26.2), 4-{[(2-Chloro1,3-thiazolo-5-yl)methyl](2-fluoroethyl)amino}furan-2(5H)-on (A.26.3), 4-{[(6-Chloropyrid-3-yl)methyl](2-fluoroehtyl)amino}furan-2(5H)-on (A.26.4), 4-{[(6-Chloropyrid-3-yl)methyl](2,2-difluoroethyl)amino}furan-2(5H)-on (A.26.5), 4-{[(6-Chloro-5-fluoropyrid-3-yl)methyl](methyl)amino}furan-2(5H)-on (A.26.6), 4-{[(5,6-Dichloropyrid-3-yl)methyl](2-fluoroethyl)amino}furan-2(5H)-on (A.26.7), 4-{[(6-Chloro-5-fluoropyrid-3-yl)methyl](cyclopropyl)amino}furan-2(5H)-on (A26.8), 4-{[(6-Chloropyrid-3-yl)methyl](cyclopropyl)amino}furan-2(5H)-on (A.26.9) and 4-{[(6-Chloropyrid-3-yl)methyl](methyl)amino}furan-2(3H)-on (A.26.10);

A.27. Various compounds selected from the group consisting of aluminum phosphide, amidoflumet, benclothiaz, benzoximate, bifenazate, borax, bromopropylate, cyanide, cycnopyrafen, cyflumetofen, chinomethionatc, dicofol, fluoroacctate, phosphine, pyridalyl, pyrifluquinazon, sulfur, organic sulfur compounds, tartar emetic, sulfoxaflor, N—R′-2,2-dihalo-1-R″cyclo-propanecarboxamide-2-(2,6-dichloro-α,α,α-trifluoro-p-tolyl)hydrazone or N—R′-2,2-di(R′″)propionamide-2-(2,6-dichloro-α,α,α-trifluoro-p-tolyl)-hydrazone wherein R′ is methyl or ethyl, halo is chloro or bromo, R″ is hydrogen or methyl and R′″ is methyl or ethyl, 4-But-2-ynyloxy-6-(3,5-dimethyl-piperidin-1-yl)-2-fluoro-pyrimidine (A.27.1), Cyclopropaneacetic acid 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3pyridinyl)-2H,11H-naphtho[2,1-b]pyrano[3,4-e]pyran-3,6-diyl]ester (A.27.2) and 8-(2-Cyclopropylmethoxy-4-trifluoromethyl-phenoxy)-3-(6-trifluoromethyl-pyridazin-3-yl)-1-aza-bicyclo[3.2.1]octane (A.27.3) in synergistically effective amounts.

Moreover, we have found that simultaneous, that is joint or separate, application of one or more active compounds I and one or more active compounds II or successive application of one or more active compounds I and one or more active compounds II allows enhanced control of pests compared to the control rates that are possible with the individual compounds.

The present invention also provides a method for controlling insects, acarids or nematodes comprising contacting the insect, acarid or nematode or their food supply, habitat, breeding grounds or their locus with a pesticidally effective amount of the mixture according to the invention.

Moreover, the present invention relates to a method for protecting growing plants from attack or infestation by insects, acarids or nematodes comprising contacting the plant, or the soil or water in which the plant is growing, with a pesticidally effective amount of the mixture according to the invention.

The present invention also provides a method for the protection of plant propagation material from pests comprising contacting the plant propagation material with a pesticidally effective amount of the mixture according to the invention.

The present invention also provides a plain propagation material, comprising the mixture according to the invention in an amount of from 0.1 g to 10 kg per 100 kg of the plant propagation material.

The present invention also relates to the use of the mixture according to the invention for combating insects, arachnids or nematodes.

The present invention also concerns the use of the mixture according to the invention for combating parasites in and on animals.

The present invention also provides a method for protecting an animal against infestation or infection by parasites or treating, an animal infested or infected by parasites which comprises orally, topically or parenterally administering or applying to the animal a parasitically effective amount of the mixture according to the invention.

### DETAILED DESCRIPTION OF INVENTION

According to the invention, the active compound I is selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains.

The strain of Streptomyces galbus having accession number NRRL 30232, its mutants, its variants, metabolites, its supernatants, and solvent extracts including their preparation, characterization, insecticidal activity and compositions comprising the same have been described in U.S. Pat. No, 6,682,925 B1.

The strain of Streptomyces galbus having accession number NRRL 30232 is also known as Streptomyces galbus strain AQ6047 from AgraQuest Inc., USA.

The strain of Streptomyces galbus having accession number NRRL 50334 is also known as Streptomyces galbus strain M1064 from AgraQuest Inc., USA.

“NRRL” is the abbreviation for the Agricultural Research Culture Collection, an international depositary authority for the purposes of deposing microorganism strains under the Budapest Treaty on the International Recognition of the Deposit of Microorganisms for the Purposes of Patent Procedure, having the address National Center for Agricultural Utilization Research, Agricultural Research Service, U.S. Department of Agriculture, 1815 North University Street, Peoria, Ill. 61604, USA.

The term “mutant” refers to as variant of the parental strain as well as methods for obtaining a mutant or variant in which the pesticidal activity is greater than that expressed by the parental strain. The “parental strain” is defined herein as the original Streptomyces strain before mutagenesis. To obtain such mutants the parental strain may be treated with a chemical such as N-methyl-N′-nitro-N-nitrosoguanidine, ethylmethanesulfone, or by irradiation using gamma, x-ray, or UV-irradiation, or by other means well known to those practiced in the art.

A “variant” is a strain having all the identifying characteristics of NRRL Accession No. 30232 and can identified having genome that hybridizes under conditions of high stringency to the genome of NRRL Accession No. B-30232.

A variant of NRRL Accession No. 30232 may also be defined as a strain having a genomic sequence that is greater than 85%, more preferably greater than 90% or more preferably greater than 95% sequence identity to the genome of NRRL Accession No. 30232. A polynucleotide or polynucleotide region (or a polypeptide or poly-peptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example, those described in Current Protocols in Molecular Biology (F. M. Ausubel et al., eds., 1987) Supplement 30, section 7.7.18, Table 7.7.1.

The term “metabolite” refers to any compound, substance or by product of the fermentation of a microorganism that has biological activity.

The active compound I embraces not only the isolated, pure cultures of the Streptomyces galbus strain, but also their suspensions in a whole broth culture or as a metabolite-containing supernatant or a purified metabolite obtained from a whole broth culture of the strain.

“Whole broth culture” refers to a liquid culture containing both cells and media. “Supernatant” refers to the liquid broth remaining when cells grown in broth are removed by centrifugation, filtration, sedimentation, or other means well known in the art.

The insecticidal activity of the aforementioned strain, its mutants, its variants and metabolites produced by said strain is, in particular at low application rates, not entirely satisfactory.

U.S. Pat. No, 6,682,925 B1 does not disclose pesticidal mixtures comprising the active compound 1 which shows unexpected and synergistic effects in combination with other pesticidically active compounds.

The commercially available active compounds II of the groups A.1 to A.27 may be found in The Pesticide Manual, 13th Edition, British Crop Protection Council (2003) among other publications.

Paraoxon and their preparation have been described in Farm Chemicals Handbook, Volume 88, Meister Publishing Company, 2001. Flupyrazofos has been described in Pesticide Science 54, 1988, p. 237-243 and in U.S. Pat. No. 4,822,779. AKD 1022 and its preparation have been described in U.S. Pat. No. 6,300,348. The anthranilamides A.23.1 to A.23.6 have been described in WO 2008/72743 and WO 200872783, those A.23.7 to A.23.12 in WO2007/043677. The phthalamide A.21.1 is known from WO 2007/101540. The alkynylether compound A.27.1 is described e.g. in JP 2006131529. Organic sulfur compounds have been described in WO 2007060839. The isoxazoline compounds A.22.1 to A.22.5 have been described in e.g., WO2005/085216. WO 2007/079162 and WO 2007/026965. The aminofuranone compounds A.26.1.to. A.26.10 have been described in WO 2007/115644. The pyripyropene derivative A.27.2 has been described in WO 2008/66153 and WO 2008/108491. The:pyridazin compound. A27.3 has been described in JP 2008/115155. Malononitrile compounds such as A.24.1. and A.24.2 have been described in WO 02/089579, WO 02/090320, WO 02/090321, WO 04/006677, WO 05/068423, WO 05/068432 and WO 05/063694.

### Preferences

### Preferred Active Compounds I

With regard to its use in the pesticidal mixtures of the present invention, the active compound I is preferably the Streptomyces galbus strain having accession number NRRL 30232 or the Streptomyces galbus strain having accession number NRRL 50334.

With regard to its use in the pesticidal mixtures of the present invention, the active compound I is preferably a pure culture of the Streptomyces galbus strain having accession number NRRL 30232 or the Streptomyces galbus strain having accession number NRRL 50334.

With regard to its use in the pesticidal mixtures of the present invention, the active compound I is preferably a mutant of the Streptomyces galbus strain having accession number NRRL 30232 or the Streptomyces galbus strain having accession number NRRL 50334, wherein said mutant has insecticidal activity.

With regard to its use in the pesticidal mixtures of the present invention, the active compound I is preferably a pure culture of a mutant of the Streptomyces galbus strain having accession number NRRL 30232 or the Streptomyces galbus strain having accession number NRRL 50334, wherein said mutant has insecticidal activity.

With regard to its use in the pesticidal mixtures of the present invention, the active compound I is preferably a variant of the Streptomyces galbus strain having accession number NRRL 30232 or the Streptomyces galbus strain having accession number NRRL 50334, wherein said variant has insecticidal activity.

With regard to its use in the pesticidal mixtures of the present invention, the active compound I is preferably a pure culture of a variant of the Streptomyces galbus strain having accession number NRRL 30232 or the Streptomyces galbus strain having accession number NRRL 50334, wherein said variant has insecticidal activity.

With regard to its use in the pesticidal mixtures of the present invention, the active compound I is preferably a metabolite produced by the Streptomyces galbus strain having accession number NRRL 30232 or the Streptomyces galbus strain having the Streptomyces galbus strain having accession number NRRL 50334, wherein said metabolite has insecticidal activity.

With regard to its use in the pesticidal mixtures of the present invention, the active compound I is preferably a supernatant obtained from the whole broth culture of the Streptomyces galbus strain having accession number NRRL 30232 or the Streptomyces galbus strain having accession number NRRL 50334, wherein said supernatant has insecticidal activity.

With regard to its use in the pesticidal mixtures of the present invention, the active compound I is preferably a solvent extract of the supernatant obtained from the whole broth culture of the Streptomyces galbus strain having accession number NRRL 30232 or the Streptomyces galbus strain having accession number NRRL 50334, wherein said solvent extract has insecticidal activity.

With regard to its use in the pesticidal mixtures of the present invention, the active compound I is preferably a whole broth culture of is the Streptomyces galbus strain having accession number NRRL 30232 or the Streptomyces galbus strain having accession number NRRL 50334, wherein said whole culture broth has insecticidal activity.

With regard to its use in the pesticidal mixtures of the present invention, the active compound I is preferably contained in a liquid or dry composition. Such liquid or dry compositions comprise the active compound I in an amount of from 1 wt. % to 100 wt. %, preferably from 75 wt. % to 100 wt. % based on the total weight composition and a carrier. The active compound I can be formulated as any one or more of a wettable powder, a granule, an aqueous suspension, an emulsifiable concentrate and a microeticapsulated formulation. Preferably, the active compound I is contained in a dry composition, in particular in a wettable powder or granule.

### Preferred Active Compounds II

With respect to the use in the pesticidal mixture of the present invention, the active compound II is preferably selected from the groups A.1, A.2, A.3, A.5, A.6,A.7, A.10, A.12, A.14, A.18, A.20, A.21, A.22, A.23, A.25, and A.27, more preferably selected from the groups A.3, A.5, A.6, A.7, A.14, A.21 and. A.23, and even more preferably selected from the group A.14.

With respect to their use in the pesticidal mixture of the present invention, particular preference is given to the active compounds II as listed in the paragraphs below.

With regard to the use in the pesticidal mixture of the present invention, the active compound II selected from group A.1 as defined above is preferably acephate, chlorpyrifos, or diazinon.

With regard to the use in the pesticidal mixture of the present invention, the active compound II selected from group A.2 as defined above is preferably carbaryl or methomyl,

With regard to the use in the pesticidal mixture of the present invention, the active compound II selected from group A.3 as defined above is preferably allethrin, bifenthrin, cyfluthrin, lambda-cyhalothrin, cypemethrin, alpha-cypermethrin, beta-cypermethrin, zeta-cypermethrin, deltamethrin, etofenprox, fenpropathrin, fenvalerate, flucythrinate, pyrethrin (pyrethrum), taufluvalinate, silafluofen or tralomethrin. More preferably, the active compound II is alpha-cypermethrin deltamethrin.

With regard to the use in the pesticidal mixture of the present invention, the active compound II selected from group A.5 as defined above is preferably thiamethoxam, spinosad or spinetoram. More preferably the active compound II is thiamethoxam or spinosad.

With regard to the use in the pesticidal mixture of the present invention, the active compound II selected from group A.6 as defined above is preferably fipronil.

With regard to the use in the pesticidal mixture of the present invention, the active compound II selected from group A.7 as defined above is preferably emamectin benzoate or lepimectin.

With regard to the use in the pesticidal mixture of the present invention, the active compound II selected from group A.10 as defined above is preferably chlorfenapyr.

With regard to the use in the pesticidal mixture of the present invention, the active compound II selected from group A.12 as defined above is preferably halofenozide.

With regard to the use in the pesticidal mixture of the present invention, the active compound II selected from group A.14 as defined above is preferably indoxacarb or metaflumizone. More preferably, the active compound II is metaflumizone.

With regard to the use in the pesticidal mixture of the present invention, the active compound II selected from group A.18 as defined above is preferably lufemuron or novahuron.

With regard to the use in the pesticidal mixture of the present invention, the active compound II is selected from group A.20, i.e., amitraz.

With regard to the use in the pesticidal mixture of the present invention, the active compound II selected from group A.21 as defined above is preferably flubendiamide.

With regard to the use in the pesticidal mixture of the present invention, the active compound II selected from group A.22 as defined above is preferably 4-[5-(3,5-Dichloro-phenyl)-5-trifluoromethyl-4,5-dihydro-isoxazol-3-yl]-2-methyl-N-pyridin-2-ylmethyl-benzamide.

With regard to the use in the pesticidal mixture of the present invention, the active compound II selected from group A.23 as defined above is preferably chloranthraniliprole or cyantraniliprole.

With regard to the use in the pesticidal mixture of the present invention, the active compound II selected from group A.25 as defined above is preferably Bacillus thuringiensis subsp. Kurstaki.

With regard to the use in the pesticidal mixture of the present invention, the active compound II selected from group A.27 as defined above is preferably pyridalyl.

With regard to the use in the pesticidal mixture of the present invention, the active compound II is preferably selected from the group consisting of acephate, chlorpyrifos, diazinon, carbaryl, methomyl, allethrin, bifenthrin, cyfluthrin, lambda-cyhalothrin, cypermethrin, alpha-cypermethrin, beta-cypermethrin, zeta-cypermethrin, deltame-thrin, etofenprox, fenpropathrin, fenvalerate, flucythrinate, pyrethrin, tau-fluvalinate, silafluofen, tralomethrin, thiamethoxam, spinosad, fipronil, emamectin benzoate, lepimectin, halofenozide, chlorfenapyr, indoxacarb, metaflumizone, lufenuron, novaluron, amitraz, flubendiamide, 4-[5-(3,5-Dichloro-phenyl)-5-trifluoromethyl-4,5-dihydro-isoxazol-3-yl]-2-methyl-N-pyridin-2-ylmethyl-benzamide, chloranthraniliprole, cyantraniliprole, Bacillus thuringiensis subsp, Kurstaki and pyridalyl.

Especially preferred are pesticidal mixtures containing acephate as active compound II.

Especially preferred are pesticidal mixtures containing chlorpyrifos as active compound II.

Especially preferred are pesticidal mixtures containing diazinon as active compound II.

Especially preferred are pesticidal mixtures containing carbaryl as active compound II.

Especially preferred are pesticidal mixtures containing methomyl as active compound II.

Especially preferred are pesticidal mixtures containing allethrin as active compound II.

Especially preferred are pesticidal mixtures containing bifenthrin as active compound II.

Especially preferred are pesticidal mixtures containing cyfluthrin as active compound II.

Especially preferred are pesticidal mixtures containing lambda-cyhalothrin as active compound II.

Especially preferred are pesticidal mixtures containing deltamethrin as active compound II.

Especially preferred are pesticidal mixtures containing cypermethrin as active compound II.

Especially preferred are pesticidal mixtures containing halofenozide as active compound II.

Especially preferred are pesticidal mixtures containing alpha-cypermethrin as active compound II.

Especially preferred are pesticidal mixtures containing beta-cypermethrin as active compound II.

Especially preferred are pesticidal mixtures containing zeta-cypermethrin as active compound II.

Especially preferred are pesticidal mixtures containing etofenprox as active compound II.

Especially preferred are pesticidal mixtures containing fenvalerate as active compound II.

Especially preferred are pesticidal mixtures containing flucythrinate as active compound II.

Especially preferred are pesticidal mixtures containing pyrethrin as active compound II.

Especially preferred are pesticidal mixtures containing taufluvalinate as active compound II.

Especially preferred are pesticidal mixtures containing silafluofen as active compound II.

Especially preferred are pesticidal mixtures containing tralomethrin as active compound II.

Especially preferred are pesticidal mixtures containing thiamethoxam as active compound II.

Especially preferred are pesticidal mixtures containing spinosad as active compound II.

Especially preferred are pesticidal mixtures containing fipronil as active compound II.

Especially preferred are pesticidal mixtures containing emamectin benzoate as active compound II.

Especially preferred are pesticidal mixtures containing lepimectin as active compound II.

Especially preferred are pesticidal mixtures containing chlorfenapyr as active compound II.

Especially preferred are pesticidal mixtures containing indoxacarb as active compound II.

Especially preferred are pesticidal mixtures containing metaflumizone as active compound II. In one embodiment, metaflumizone is contained in a suspension concentrate (SC).

Especially preferred are pesticidal mixtures containing lufenuron as active compound II.

Especially preferred are pesticidal mixtures containing novaluron as active compound II. Especially preferred are pesticidal mixtures containing amitraz as active compound II.

Especially preferred are pesticidal mixtures containing flubendiamide as active compound II.

Especially preferred are pesticidal mixtures containing 4-[5-(3,5-Dichloro-phenyl)-5-trifluoromethyl-4,5-dihydro-isoxazol-3-yl]-2-methyl-N-pyridin-2-ylmethyl-benzamide as active compound II.

Especially preferred are pesticidal mixtures containing chloranthraniliprole as active compound II.

Especially preferred are pesticidal mixtures containing cyantraniliprole as active compound II.

Especially preferred are pesticidal mixtures containing Bacillus thuringiensis subsp. Kurstaki as active compound II.

Especially preferred are pesticidal mixtures containing pyridalyl as active compound II.

### Preferred Mixtures According to the Invention

Especially preferred are inventive mixtures wherein the compound I is the Streptomyces galbus strain having accession number NRRL 30232 or the Streptomyces galbus strain having accession number NRRL 50334 and the active compound II is metaflumizone.

### Pests

The mixtures of the active compounds I and II, or the active compounds I and II used simultaneously, that is jointly or separately, exhibit outstanding action against invertebrate pests.

In the sense of the present invention, the invertebrate pests are preferably selected from arthropods and nematodes, more preferably from insects, arachnids and nematodes, and even more preferably from insects, acarids and nematodes.

The mixtures of the present invention are especially suitable for efficiently combating the following invertebrate pests: insects from the order of the lepidopterans (Lepidoptera), for example, Agrotisypsilon, Agrotis segetum, Alabama argillaeca, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Choristoneura rosaceana, Cirphis unipuncta, Cydia pomonella, Dendrolimus pini, Diaphana nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana Loxostege sticticalis, Lymantria dispar, Lymantria monacha, Lyonetia clerkella, Malacosoma neustria, Mamestra brassicae, Orgyia pseudotsugata, Ostrinia nubilalis, Pandemis pyrusana, Panolis flammea, Pectinophora gossypiella, Peridroma saucia, Phalera bucephala, Phthorimaca operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera eridania, Spodoptera exigua, Spodoptera frugiperda, Spodoptera littoralis, Spodoptera litura, Thaumatopoea pityocampa, Tortrix viridana, Trichoplusia ni and Zeiraphera canadensis, beetles (Coleoptera), for example, Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus pimiperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuhorrhynchus napi, Chaetocnema tibialis, Conoderus vespertinus, Crioceris asparagi, Ctenicera ssp., Diabrotica longicomis, Diabrotica semipunetata, Diabrotica 12-punctata Diabrotica speciosa, Diabrotica virgifera, Epilachna varivestis, Epitrix hirtipennis, Eutinobothrus brasiliensis, Hylobins abietis, Hypera brunneipennis, Hypera postica, Ips typographus, Lema bilineata, Lema melanopus, Leptinotarsa decemlineata, Limonius califormieus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phylloterta striolata, Popillia japonica, Sitona lineatus and Sitophilus granaria, flies, mosquitoes (Diptera), e.g., Aedes aegypti, Acdes albopictus, Acdes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivoras, Contarinia sorghicola Cordylobia anthropophaga, Culicoides furens, Culex pipiens, Culex nigripalphus, Culex quinquefasciatus, Culex tarsalis, Culiseta inornata, Culiseta melanura, Dacus cucurbitae, Dacus oleae, Dasineura brassicae, Delia antique, Delia coarciata, Delia platura, Delia radicum, Dermatobia hominis, Fannia canicularis, Geomyza Tripunctata, Gasterophilis intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineate, Leptoconops torrens, Lirimyza sativae, Liriomyza trifolii, Lucilia caprina, Lucilia cuprina, Lucilia sericata, Lycoria pectoralis, Mansonia titillanus, Mayeoiola destructor, Musca autummalis, Musca domestica, Muscina stabulans, Oestrus ovis, Opomyza florum, Oscinella frit, Pegonya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis Sarcophaga spp., Simulium vittatum, Stomoxys calcitrans, Tabanus bovinus, Tabanus atratus, Tabanus lineola, and Tabanus similis, Tipula oleracea, and Tipula paludosa thrips (Thysanopthera), e.g., Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci, termites (Isoptera), e.g., Calotermes flavicollis, Leucotermes flavipes, Heterotermes aureus, Reticulitermes flavipes, Reticulitermes, virginicus, Reticulitermes lucifugus, Reticulitermes santonensis, Reticulitermes grassei, Termes natalensia, and Coptotermes formosanus, cockroaches (Blattaria-Blattodea), e.g., Blattella germanica, Blattella asahinae, Periplaneta americana, Periplaneta japonica, Periplaneta brunnea, Periplaneta fuligginosa, Periplaneta australasiae, and Blatta orientalis, bugs, aphids, leafhoppers, whiteflies, scale insects, cicadas (Hemiptera), e.g., Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lincolaris, Lygus pratensis, Nezara viridula, Piesma quadrata, Soluben insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneider, Aphis spiraccola, Aphis sambuci, Acyrthosiphon pisum, Aulacorthum solari, Bemisia argentifolii, Brachycaudus cardui, Brachycaudus helichrysi, Brachycaudus persicae, Brachycaudus prunicola, Brevicoryne brassicae, Capitophorus homi, Cerosipha gossypii, Chaetosiphon fragaefolii, Cryptomyzus ribis, Dreyfusia nordmannianae, Dreyfusia piceae, Dysaphis radicola, Dysaulacorthaum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Nyzus varians, Nasonivia ribinigri, Nilaparvata lugens, Pemphigus bursaris, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis gramimum, Schizoneura lanuginosa, Sitobion avenae, Trialeurodes vaporarirum, Toxoptera auranuiand, Viteus vitifolii, Cimex lectularius, Cimex hemipterus, Reduvius senilis, Triatoma spp., and Arilus critatus, ants, bees, wasps, sawflies (Hymenoptera), e.g., Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbaus, Pogonomyrmex californicus, Pheidole megacephala, Dasymutilla occidentalis, Bombus spp., Vespula squamosa, Paravespula vulgaris, Paravespula pennsylvanica, Paravespula gemanica, Dolichovespula maculata, Vespa crabro, Polistes rubiginosa, Camponotus floridanus, and Linepithema humile, crickets, grasshoppers, locusts (Orthoptera), e.g., Acheta domestica, Gryllotalpa gryllotalpa, Locusta migratoria, Melanoplus bivittatus, Melanoplus Femurrubrum, Melanoplus mexicanus, Melanoplus sanguinipes, Melanoplus spretus, Nomadacris septemfasciata, Schistocerca american, Schistocerea gregaria, Dociostaurus marocanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina, Arachnoide, such as arachnids (Acarina), e.g., of the familes Argasida, Ixodidae and Sarcoptidae, such as Amblyomma americanum, Amblyomma variegatum, Ambryomma maculatum, Argas persicus, Boophilus annulatus, Boophilus decolaratus, Boophilus microplus, Dermacentor silvarum, Dermacentor andersomi, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Oiobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp., such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g., Latrodectus mactans, and Loxosceles reclusa, fleas (Siphonaptera), e.g., Ctenocephalides felis, Ctenocephalides canis, Xenopsylla cheopis, Pulex irritans, Tunga penetrans, and Nosopsyllus fasciatus, silverfish, firebrat (Thysanura), e.g., Lepisma saccharina and Thermobia domestica, centipedes(Chilopoda), e.g., Scutigera coleoptrata, millipedes (Diplopoda), e.g., Narceus spp., Earwigs (Dermaptera), e.g., forficula auricularia, lice (Phthiraptera), e.g., Pediculus humanus capitis, Pediculus humanus corporis, Pthirus pubis, Haematopinus eurysternus, Haematopinus suis, Linognatbus vituli, Bovicola bovis, Menopon gallinae, Menacanthus stramineus and Solenopotes capillatus, Plant parasitic nemotodes such as root-knot nematodes, Meloidogyne arenaria, Meloidogyne chitwoodi, Meloidogyne exigua, Meloidogyne hapla, Meloidogyne inognita, Meloidogyne javanica and other Meloidogyne species; cyst nematodes, Globodera rostochiensis, Globodera pallida, Globodera tabacum and other Globoder species, Heterodera avenae, Heterodera glycines, Heterodera schachtii, Heterodera trifolii, and other Heterodera species: seed gall nematodes, Anguina funesta, Anguina tritici and other Anguina species; stem and foliar nematodes, Aphelenchoides besscyi, Aphelenchoides fragarie, Aphelenchoides ritzemabosi and other Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicoty lenchus dihystera, Helicotylenchus muliticinctus and other Helicotylenchus species. Rotylenchus robustus and other Rotylenchus species; sheath nematodes, Hemicycliphora species and Hemicriconemoides and species; Hirshmanniella species; lance nematodes, Hoplolaimus columbus Hoplolaimus galeatus and other Hoplolaimus species; false root-knot nematodes, Nacobbus aberrans and other Nacobbus species; needle nematodes, Longidorus elongates and other Longidorus and species; pin nematodes, Paratylenchus species; lesion nematodes, Pratylenchus brachyurus Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, Radopholus similis and other Radopholus species; reniform nematodes, Rotylenchulus reniformis and other Rotylenchulus species; Scutellonema species stubby root nematodes, Trichodorus primitivus and other Trichodorus species; Paratrichodorus minor and other Paratrichodorus species; stunt nematodes, Tylenchorhynchus claytoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species; and other plant parasitic nematode species.

Moreover, the mixtures of the present invention are especially useful for controlling insects, or arachnids, in particular insects selected from the group consisting of Lepidoptera, Coleoptera and Diptera and arachnids of the order Acarina. The mixtures of the present invention are particularly useful for controlling insects selected from the group consisting of Lepidoptera, Coleoptera and Diptera.

In one embodiment, the inventive mixtures are useful for the control of foliar insect pests. Preferably, the foliar insect pest is a species of the order Lepidoptera.

In particular, the inventive mixtures are useful for the control of Lepidoptera.

Preferably, the insect of the order Lepidoptera is selected from the group consisting of the families Noctuidae, Plutellidae and Tortricidae.

Preferably, the insect of the order Lepidoptera is selected from the family Noctuidae, Preferably, the insect of the family Noctuidae is selected from the group consisting of Spodoptera eridania, Spodoptera exigua, Anticarsia gemmatalis, Helicoverpazea, Heliothis virescens, Spodoptera littoralis, Spodoptera frugiperda, Agrotis ipsilon, and Trichoplusia ni. In particular, the insect of the family Noctuidae is Spodoptera cridatlia.

Preferably, the insect of the family Plutellidae is Plutella xylostella.

Preferably, the insect of the family Tortricidae is selected from the group consisting of Choristoneura rosaceana, Pandemis pyrusana and Cydia pomonella.

In another embodiment of this invention, the inventive mixtures are useful for the control of Lepidoptera selected from the group consisting of Spodoptera eridania, Spodoptera exigua, Anticarsia gemmatalis, Pluteria xylostella, Helicaverpa zea, Heliothis virescens, Spodoptera littoralis, Spodoptera frugiperda, Choristoneura rosaceana, Agrotis Pandemis pyrusana, Cydia pomonella and Trichoplusia ni. Preferably, the Lepidoptera is Spodoptera eridania.

### Formulations

The pesticidal mixtures according to the present invention cart be converted into the customary formulations, e.g., solutions, emulsions, suspensions, dusts, powders, pastes, granules and directly sprayable solutions. The use form depends on the particular purpose and application method. Formulations and application methods arc chosen to ensure in each case a fine and uniform distribution of the active compounds according to the invention.

The formulations are prepared in a known manner (see e.g., for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB Patent No. 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2, D. A. Knowles, Chemistry and Technology of Agrochemical Formulations, Kluwer Academie Publishers, Dordrecht, 1998 (ISBN 0-7514-0443-8), for example, by extending the active compound with auxiliaries suitable for the formulation of agro-chemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Examples of suitable solvents/carriers are e.g.:
solvents such as water, aromatic solvents (for example, Solvesso products, xylene and the like), paraffins (for example mineral fractions), alcohols (for example methanol, butanol, pentatiol, benzyl alcohol), ketones (for example cycloheximone, gamma-butyrolactone), pyrrolidones (N-methyl-pyrrolidone (NMP),N-octylpyrrolidone NOP), acetates (glycol diacetate), alkyl lactates, lactones such as g-butyrolactone glycols, fatty acid dimethylamides, fatty acids and fatty acid esters, triglycerides, oils of vegetable or animal origin and modified oils such as alkylated plant oils. In principle, solvent mixtures may also be used.
carriers such as around natural minerals and ground synthetic minerals, such as silica gets, finely divided silicic acid, silicates, talc, kaolin, attaclay, limestone, lime, chalk, bole, loess, clay, dolomite, diatomaceous earth, calcium sulfate and magnesium sulfate, matmesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phcnolsulfonic acid, dibutylnaphthalenesulfonic acid, alkylarylsulfonates, alkyl sulfates, alkylsulfonates, fatty alcohol sulfates, fatty acids arid sulfated fatty alcohol glycol ethers, furthermore condensates of sulfonated naphthalene and naphthalene derivatives with formaldehyde, condensates of naphthalene or of nophibtilenesulfenie acid with phenol and formaldehyde, polyoxyethylene octylphenyl ether, ethoxylated isooctylphenol, octylphenol, nonylphenol, alkylphenyl polyglycol ethers, tributylphenyl palyglyeol ether, tristearylphonyl polyglycol ether, alkylaryl polyether alcohols, alcohol and fatty alcohollethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Also and-freezing agents such as glycerin, ethylene propylene glycol and bactericides such as can be added to the formulation.

Suitable antifoaming agents are for example antilbaming agents based on silicon or magnesium stearate.

Suitable preservatives are for example dichlorophen and benzyl alcohol hemiformal. Suitable thickeners are compounds which confer a pseudoplastic flow behavior to the formulation, i.e., high viscosity at rest and low viscosity in the agitated stage.

Mention may be made, in this context, for example, of commercial thickeners based on polysaccharides, such as Xanthan Gum® (Kelzan® from Kelco), Rhodopol®23 (Rhone Poulene) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kothon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, botanic acid, acetic acid, propionic acid, citric acid, fumarie acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphtlialene, alkylated naphthalenes or their derivatives, methanol, ethanol, propanol, butanol, cyclohexanol, cyclohexanone, isophorone, strongly polar solvents, for example dimethyl sulfoxide, methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds II are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The mixtures of the present invention can be used as such, in the form of their formulations or the use forms prepared therefrom, for example, in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following arc examples or formulations:

Products for dilution with water. For seed treatment purposes, such products may be applied to the seed diluted or undiluted.

A) Water-Soluble Concentrates (SL, LS)

10 parts by weight of the active compound(s) is dissolved in 90 parts by weight of water or a water-soluble solvent. As an alternative, welters or other auxiliaries are added. The active compound(s) dissolve(s) upon dilution with water, whereby a formulation with 10% (w/w) of active compound(s) is obtained.

B) Dispersible Concentrates (DC)

20 parts by weight of the active compound(s) is dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

C) Emulsifiable Concentrates (EC)

15 parts by weight of the active compound(s) is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

D) Emulsions (EW, EO, ES)

25 parts by weight of the active compound(s) is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g., Ultraturrax) and made into a homogeneous emulsion. Dilution with water gives an emulsion, whereby a formulation with 25% (w/w) of active eompound(s) is obtained.

E) Suspensions (SC, OD, FS)

In an agitated ball mill, 20 parts by weight of the active compound(s) is comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

F) Water-Dispersible Granules and Water-Soluble Granules (WG, SG)

50 parts by weight of the active compound(s) is ground finely with addition of 50 parts by weight or dispersants and wetters and made as water-dispersible or water-soluble granules by means of technical appliances (for example, extrusion, spray tower, fluidized bed). Dilution with water gives a stable dispersion or solution or the active compound(s), whereby a formulation with 50% (w/w) of active compound(s) is obtained.

G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

H) Gel-Formulation (GF)

In an agitated ball mill, 20 parts by weight of the active compound(s) is comminuted with addition of 10 pans by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active eompound(s) is obtained.

2. Products to be applied undiluted for foliar applications. For seed treatment purposes, such products may be applied to the seed diluted or undiluted.

I) Dustable Powders (DP, DS)

5 parts by weight of the active compound(s) are ground finely and mixed intimately with 95 parts by weight of finely divided kaolin. This gives a dustable product having 5% (w/w) of active compound(s).

J) Granules (GR, FG, GG, MG) 0.5 part by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

K) ULV solutions (UL)

10 parts by weight of the active compound(s) is dissolved in 90 parts by weight of an organic solvent, for example xylene. This gives a product having 10% (w/w) of active compound(s), which is applied undiluted for foliar use.

Aqueous use forms can be prepared from emulsion concentrates, pastes or wettable powders t:sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate solvent or oil, and such concentrates are suitable for dilution with water.

The concentrations of the active compound(s) in the ready-to-use products can be active ingredient, or even to apply the active ingredient without additives.

Various types of oils, wetters, adjuvants, herbicides, fungicides, other pesticides, or bactericides may be added to the active compounds, if appropriate just immediately prior to use (tank mix). These agents usually are admixed with the active compounds according to the invention in a weight ratio of 1:10 to 10:1.

### Applications

The active compounds I and used in the pesticidal mixtures according to the invention can be applied according to different ways of applications, e.g.,

A) simultaneously, that is

a1) jointly (i.e. as mixture as such, e.g., a ready-to-use-formulation, or as tank mix) or

a2) separately (i.e. application via separate tanks), or

B) in succession, the sequence, in this case, generally not having any effect on the result of the control measures.

The pesticidal mixtures of this invention, in particular being present in form of compositions of this invention, may further contain other active ingredients than those listed above, for example fungicides, herbicides, fertilizers such as ammonium nitrate, urea, potash, and superphosphate, phytotoxicants and plant growth regulators and safeners. These additional active ingredients may be used sequentially or in combination with the above-described compositions, if appropriate also added only immediately prior to use (tank mix). For example, the plant(s) may be sprayed with a composition of this invention either before or after being treated with other active ingredients.

The pesticidal mixtures of this invention may be prepared by any suitable methods, for instance by mixing all of the components, e.g., active compounds, or by preparing a pre mixture of two or more active compounds and then adding further components, e.g., other active ingredient(s).

The one or more active compound(s) I and the one or more active compound(s) II are usually applied in a weight ratio of from 600:1 to 1:100, preferably from 600:1 to 1:50, in particular from 600:1 to 1:20. Depending on the desired effect, the application rates of the pesticidal mixtures according to the invention are from 1 g/ha to 2000 g/ha, preferably from 2.5 to 1500 g/ha.

The invertebrate pest, e.g., the insects, arachnids and nematodes, the plant, soil or water in which the plant is growing can be contacted with the pesticidal mixtures of this invention or composition(s) containing them by any application method known in the art. As such, “contacting” includes both direct contact (applying the mixtures/compositions directly on the animal pest or plant—typically to the foliage, stem or roots of the plant) and indirect contact (applying the mixtures/compositions to the locus of the invertebrate pest or plant).

The pesticidal mixtures of this invention or the pesticidal compositions comprising them may be used to protect growing plants and crops from attack or infestation by invertebrate pests, especially insects, acaridae or arachnids by contacting the plant/crop with a pesticidally effective amount of the active compounds I and II. The term “crop” refers both to growing and harvested crops.

In one embodiment, the pesticidal mixture of this invention or the pesticidal composition comprising them is sprayed onto the plant or crop.

The pesticidal mixtures of this invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In one embodiment, the plant:or crop is selected from beans and pepper.

The pesticidal mixtures of this invention are employed as such or in form of compositions by treating the insects or the plants, plant propagation materials, such as seeds, soil, surfaces, materials or rooms to be protected from insecticidal attack with insecticidally effective amount of the pesticidal mixtures of this invention. The application can be carried out both before and after the infection of the plants, plant propagation materials, such as seeds, soil, surfaces, materials or rooms by the insects.

The present invention also includes a method of combating invertebrate pests which comprises contacting the invertebrate pests, their habit, breeding ground, food supply, cultivated plants, seed, soil, area, material or environment in which the invertebrate pests are growing or may grow, or the materials, plants, seeds, soils, surfaces or spaces to be protected from pest attack or infestation with a pesticidally effective amount of the pesticidal mixtures of this invention.

Moreover, invertebrate pests may be controlled by contacting the target pest, its food supply, habitat, breeding ground or its locus with pesticidally effective amount of the pesticidal mixtures of this invention. As such, the application may be carried out before or after the infection of the locus, growing crops, or harvested crops by the pest.

The pesticidal mixtures of this invention can also be applied preventively to places at which occurrence of the pests is expected.

The pesticidal mixtures of this invention may be also used to protect growing plants front attack or infestation by pests by contacting the plant with a pesticidally effective amount of the pesticidal mixtures of this invention. As such, “contacting” includes both direct contact (applying the mixtures/compositions directly on the pest and/or plant—typically to the foliage, stem or roots of the plant) and indirect contact (applying the mixtures/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e.g., potatoes), which can be used for the multiplication of the plant.

This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the Use of recombinant DNA techniques that under natural circumstances cannot readily be obtained by cross breeding., mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides) poly for example, by glycosylation or polymer additions such as prenylated, acetylated farnesylated moieties or PEG moieties (e.g., as disclosed in Biotechnol Prog. 2001 July-August; 17(4):720-8., Protein Eng Des Sel. 2004 January; 17(1):57-66, Nat Protoc. 2007; 2(5):1225-35., Curr Opin Chem Biol. 2006 October; 10(5):487-91, Epub 2006 Aug. 28., Biomaterials 2001 March; 22(5):405-17, Bioconjug Chem, 2005 January-February; 16(1):113-21).

The term “cultivated plants” is to be understood also including plants that have been rendered tolerant to applications specific classes of herbicides, such as hydroxyphenylpyruvate dioxygenase (HPPD) inhibitors; acetolactate synthase (ALS) inhibitors, such sulfonyl ureas (see e.g., U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/160731; enolpyruvylshikimate-3-phosphate synthase (EPSPS) inhibitors, such as glyphosate (see e.g., WO 92/00377); glutamine synthetase (GS) inhibitors, such as glufosinate (see e.g., EP-A-0242236, EP-A-242246) or oxynil herbicides (See e.g., U.S. Pat. No. 5,559,024) as a result of conventional methods of breeding or genetic engineering. Several cultivated plants have been rendered tolerant to herbicides by conventional methods of breeding (mutagencsis), for example Clearfield® summer rape (Canola) being tolerant to imidazolinones, e.g., imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as a-endotoxins, e.g. CryIA(b), CryIA(c), CryIF, CryIF(a2), CryIIA(b), CryIIIA, CryIIIB(b1) or Cry9c; vegetative insecticidal proteins (VIP). e.g., VIP1, VIP2, VIP3 or VIP3A; insecticidal proteins of bacteria colonizing nematodes, for example Photorhabdus spp, or Xenorhabdux spp., toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serinc protease inhibitors, patatin, cystatin or papain inhibitors; ribosome-inactivating proteins (RIP), such as ricin, maize-RIP, abrin, luffin, saporin bryodin; steroid metabolism enzymes, such as 3-hydroxysteroid oxidase, ecdysteroid-IDP-glycosyl-transirase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are disclosed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes for example potato cultivars, which express resistance genes acting against Phytophthora infestans derived from the mexican wild potato Solanum bulbocastanum) or T4-lysozym (e.g., potato cultivars capable of synthesizing these proteins with increased resistance against bacteria such as Erwiuia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g., bio mass production, grain yield, starch content, oil content or protein content), tolerance to drought, salinity or other growth-limiting environmental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for example, oil crops that produce health-promoting long-chain omega-3 fatty acids or unsaturated omega-9 fatty acids (e.g., Nexera® rape). The term “cultivated plants” is to be understood also including plants that contain by the use of recombinant DNA techniques a modified amount of substances of content or new substances of content, specifically to improve raw material production, for example, potatoes that produce increased amounts of amylopectin (e.g., Amflora® potato).

In general, “pesticidally effective amount” means the amount of active compound needed to achieve an observable effect on growth, including the effects of necrosis, death, retardation, prevention, and removal, destruction, or otherwise diminishing the occurrence and activity of the target organism. The pesticidally effective amount can vary for the various mixtures/compositions used in the invention. A pesticidally effective amount of the mixtures/compositions will also vary according to the prevailing conditions such as desired pesticidal effect and duration, weather, target species, locus, mode of application, and the like.

In the ease of soil treatment or of application to the pests dwelling place or nest, the quantity of the mixture of active compounds ranges from 0.0001 to 500 g per 100 m2, preferably from 0.001 to 20 g per 100 m2.

Customary application rates in the protection of materials are, for example, from 0.01 g to 1000 g of the mixture of active compounds per m2 treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the mixture of active compounds may be in the range of 0.1 g to 4000 g per hectare, desirably from 25 g to 600 g per hectare, more desirably from 50 g to 500 g per hectare.

The pesticidal mixtures of the present invention are effective through both contact (via soil, glass, wall, bed net, carpet, plant parts or animal parts), and ingestion (bait, or plant part).

The pesticidal mixtures of this invention may also be applied against non-crop insect pests, such as ants, termites, wasps, flies, mosquitoes, crickets, or cockroaches. For use against said non-crop pests, the pesticidal mixtures of the invention are preferably used in a bait composition.

The bait can be a liquid, a solid or a semisolid preparation (e.g., a gel). Solid baits can be formed into various shapes and forms suitable to the respective application e.g., granules, blocks, sticks, disks. Liquid baits can be filled into various devices to ensure proper application, e.g., open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitoes, crickets etc., or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of the mixture of active compounds is from 0.001 weight % to 15 weight %, desirably from 0.001 weight % to 5% weight % of the composition.

Formulations of the mixtures of this invention as aerosols (e.g., in spray cans), oil sprays or pump sprays are hi tthiy suitable for the non-professional user for controlling pests such as flies, fleas, ticks, mosquitos or cockroaches. Aerosol recipes are preferably composed of the active compound, solvents such as lower alcohols (e.g., methanol, ethanol, propanol, butanol), ketones (e.g., acetone, methyl ethyl ketone), paraffin hydrocarbons (e.g., kerosenes) having boiling ranges of approximately 50 to 250° C. dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of the mixture of active compounds is from 0.001 to 80 weights %, preferably from 0.01 to 50 weight % and most preferably from 0.01 to 15 weight %.

The mixture of this invention and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g., malaria, dengue and yellow fever, lymphatic filariasis, and leishmaniasis) with the mixtures of this invention and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, non-wovens, netting material or foils and tarpaulins preferably comprise a mixture including the insecticide, optionally a repellent and at least one binder. Suitable repellents, for example, are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected, for example from polymers and copolymers of vinyl esters of aliphatic acids (such as such as vinyl acetate and vinyl versatate), acrylic and methacrylic esters of alcohols, sueh as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The mixtures of this invention and their compositions can be used for protecting wooden materials such as trees, board fences, sleepers, etc., and buildings such as houses, outhouses, factories, but also construction materials, furniture, leathers, fibers, vinyl articles, electric wires and cables etc., from ants and/or termites, and for controlling ants and termites from doina harm to crops or human being (e.g., when the pests invade into houses and public facilities). The mixtures of this invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc., and vinyl articles such as coated electric wires, vinyl sheets, heat insulating material such as styrene foams, etc. In case of application against ants doing harm to crops or human beings, the mixtures of the present invention are applied to the crops or the surrounding soil, or are directly applied to the nest of ants or the like.

### Seed Treatment

The mixtures of this invention are also suitable for the treatment of seeds in order to protect the seed from insect pest, in pathcular from soil-living insect pests and the resulting plant's roots and shoots against soil pests and foliar insects.

The mixtures of this invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore provides a method for the protection of seeds from insects, in particular from soil insects and of the seedlings' roots and shoots from insects, in particular from soil and foliar insects, said method comprising contacting the seeds before sowing and/or after pregermination with a mixture of this invention. Particularly preferred is a method, wherein the plant's roots and shoots are protected, more preferably a method, wherein the plant's shoots are protected form piercing and sucking insects, most preferably a method, wherein the plant's shoots are protected from aphids.

The term “seed” embraces seeds and plant propagules of all kinds including but not limited to true seeds, seed pieces, suckers, corns, bulbs, fruit, tubers, grains, cuttings, cut shoots and the like and means in a preferred embodiment true seeds.

The term “seed treatment” comprises all suitable seed treatment techniques known in the art, such as seed dressing, seed coating, seed dusting, seed soaking and seed pelleting.

The present invention also provides seeds coated with or comprising the active compounds used in the mixture of this invention.

The term “coated with and/or comprising” generally signifies that the active compounds are for the most part on the surthce of the plant propagation material at the time of application, although a greater or lesser part of the active compounds may penetrate into the plant propagation material, depending on the method of application. When the said plant propagation material is (re)planted, it may absorb the active compounds.

Suitable seeds are seeds of cereals, root crops, oil crops, vegetables, spices, ornamentals, for example, seed of durum and other wheat, barley, oats, rye, mai.ze (fodder maize and sugar maize/sweet and field corn), soybeans, oil crops, crucifers, cotton, sunflowers, bananas, rice, oilseed rape, turnip rape, sugarbeet, fodder beet, eogplants, potatoes, grass, lawn, turf, fodder grass, tomatoes, leeks, ptimpkinisquash, cabbage, iceberg lettuce, pepper, cucumbers, melons, Brassica species, melons, beans, peas, garlic, onions, carrots, tuberous plants such as potatoes, sugar cane, tobacco, grapes, petunias, geraniun/pelargoniums, pansies and impatiens.

In addition, the mixtures of this invention may also be used for the treatment seeds from plants, which tolerate the action of herbicides or fungicides or insecticides owing to breeding, including genetic engineering methods.

For example, the mixtures of this invention can be employed in the treatment of seeds from plants, which are resistant to herbicides from the group consisting of the sulfonylureas, imidazolinones, glufosinate-ammonium or glyphosate-isopropylammonium and analogous active substances (see, for example, EP-A-0242236, EP-A242246) (WO 92/00377) (EP.A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259).

Furthermore, the mixtures of this invention can be used also for the treatment of seeds from plants, which have modified characteristics in comparison with existing plants consist, which can be generated, for example, by traditional breeding methods and/or the generation of mutants, or by recombinant procedures). For example, a number of cases have been described of recombinant modifications of crop plants for the purpose of modifying the starch synthesized in the plants (e.g., WO 92/11376, WO 92/14827, WO 91/19806) or of transgenic crop plants having a modified fatty acid composition (WO 91/13972).

The seed treatment application of the mixtures of this invention is carried out by spraying or by dusting the seeds before sowing of the plants and before emergence of the plants.

Compositions which are especially useful for seed treatment are e.g.:

| | |
|---|---|
| A | Soluble concentrates (SL, LS) |
| D | Emulsions (EW, EO, ES) |
| E | Suspensions (SC, OD, FS) |
| F | Water-dispersible granules and water-soluble granules (WG, SG) |
| G | Water-dispersible powders and water-soluble powders (WP, SP, WS) |
| H | Gel-Formulations (GF) |
| I | Dustable powders (DP, DS) |

Conventional seed treatment formulations include for example flowable concentrates, FS, solutions LS, powders for dry treatment DS, water dispersible powders for slurry treatment slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active compounds, 1-200 g/l surfactant, 0 to 200 g/l antifreezing agent, 0 to 400 g/l of binder, 0 to 200 g/l of a pigment and up to 1 liter of a solvent, preferably water.

Especially preferred FS formulations of the mixtures of this invention for seed treatment usually comprise from 0.1 to 80% by weight (1 to 800 g/l) of active compounds, from 0.1 to 20% by weight (1 to 200 g/l) of at least one surfactant, e.g., 0.05 to 5% by weight of a wetter and from 0.5 to 15% by weight of a dispersing agent, up to 20% by weight, e.g., from 5 to 20% of an anti-freeze agent, from 0 to 15% by weight, e.g., 1 to 15% by weight of a pigment and/or a dye, from 0 to 40% by weight, e.g., 1 to 40% by weight of a binder (sticker/adhesion agent), optionally up to 5% by weight, e.g., from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylenevinyl acetate copolymers, acrylic homo- and copolymers, polyethyleneamines, polyethyleneamides and polyethyleneimincs, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

### Examples of a Gelling Agent is Carrageen (Satiagel®)

In the treatment of seed, the application rates of the active compound(s) are generally from 0.1 g 10 kg per 100 kg of seed, preferably from 1 g to 5 kg per 100 kg of seed, more preferably 1 g to 1000 g per 100 kg of seed and in particular from 1 g to 200 g per 100 kg of seed.

The invention therefore also relates to seed comprising the mixture of this invention as defined herein. The amount of the active compound(s) will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The pesticidal mixtures of this invention are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of the pesticidal mixtures of this invention and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a pesticidal mixture of this invention or a composition comprising it.

The present invention also provides a non-therapeutic method for treating, controlling, preventing and protecting animals against infestation and infection by parasites, which comprises applying to a locus a parasiticidally effective amount of a pesticidal mixture of this invention or a composition comprising it.

The invention also provides a process for the preparation of a composition for treating, controlling, preventing or protecting animals against infestation or infection by parasites which comprises including a parasiticidally effective amount of a pesticidal mixture of this invention in a composition comprising it.

The invention relates further to the use of the pesticidal mixture of this invention or a composition comprising it for treating, controlling, preventing or protecting animals against infestation or infection by parasites.

The invention relates also to the use of the pesticidal mixtures of this invention, or a composition comprising it, for the manufacture of a medicament for the therapeutic treatment of animals against infections or infestions by parasites.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that the mixtures of this invention are suitable for combating endo- and ectoparasites in and on animals. The mixtures of this invention or the enantiomers or veterinarily acceptable salts thereof and compositions comprising them are suitable for systemic and/or non-systemic control of ecto- and/or endoparasites. They are active against all or some stages of development.

The mixtures of this invention and compositions comprising them are preferably used for controlling and preventing infestations and infections in and on animals including warm-blooded animals (including humans) and fish. They are for example suitable for controlling and preventing infestations and infections in and on mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

The mixtures of this invention and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chig-gers, gnats, mosquitoes and fleas.

The mixtures of this invention and compositions comprising them are especially useful for combating ectoparasites.

The mixtures of this invention and compositions comprising them are especially useful for combating endoparasites.

The mixtures of this invention and compositions comprising them are especially useful for combating parasites of the following orders and species, respectively: fleas (Siphonaptera), e.g., Ctenocephalides felis, Ctenocephalides canis, Xenopsylla cheopis, Pulex irritans, Tunga penetrans, and Nosopsyllus fasciatus; cockroaches (Blattaria-Blattodea), e.g., Blattella germanica, Blattella asahinae, Periplaneta americana, Periplaneta japonica, Periplaneta brunnea, Periplaneta fuligginosa, Periplaneta australasiae, and Blatta orientalis; flies, mosquitoes (Diptera), e.g., Acedes aegypti, Acdes albopictus, Acdes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacca, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorphora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lincola and Tabanus similis; lice (Phthiraptera), e.g., Pediculus humanus capitis, Pediculus humanus corporis, Pthirus pubis, Haematopinus eurystemus, Haematopinus suis, Linognathus vituli, Bovicola bovis, Menopon gallinae, Menacanthus stramineus and Solenopotes capillatus; ticks and parasitic mites (Parasitiformes); ticks (Ixodida), e.g., Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesotigmata), e.g., Ornithonyssus bacoti and Dermanyssus gallinae; Actinedida (Prostigmata) and Acaridita (Astigmata) e.g., Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp, and Laminosioptes spp; Bugs (Heteropterida); Cimex lectularius, Cimex hemipterus, Reduvius senilis, Triatoma spp., Rhodnius ssp., Panstrongylus ssp. and Arilus critatus; Anoplurida, e.g., Haematopinus spp., Linognathus spp., Pediculus spp., Phtitrus spp., and Solenopotes spp.; Mallophagida (suborders Arnblycerina and Ischnocerina), e.g., Trimenopon spp., Menopon spp., Trinoton spp., Bovicola spp., Werneckiella spp., Lepikentron spp., Trichodectes spp. and Felicola spp.; Roundworms Nematoda: Wipeworms and Trichinosis (Trichosyringida), e.g., Trichinellidae (Trichinella spp.), (Trichuridae) Trichuris spp., Capillaria spp; Rhabditida, e.g. Rhabditis spp. Strongyloides spp., Helicephalobus spp.; Strongylida, e.g., Strongylus spp., Ancylostoma spp., Necator americanus Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp., Aleurostrongylus abstrusus and Dioctophyma renale; Intestinal roundworms (Ascaridida), e.g., Ascaris lumbricoides, Ascaris suum, Ascaridia galli, Parascaris equorum, Enterobius vermicularis (Threadworm), Toxocara canis, Toxascaris leonine, Skrjabinema spp. and Oxyuris equi, Camallanida, e.g., Dracunculus medinensis (guinea worm) Spirurida, e.g., Thelazia spp. Wuchereria spp., Brugia spp., Onchocerca spp., Dirofilari spp.a, Dipetalonema spp., Setaria spp., Elacophora spp., Spirocerea lupi and Habronema spp., Thorny headed worms (Acanthocephala), e.g., Acanthocephalus spp., Macracanthorhynchus hirudinaccus and Oncicola spp, Planarians (Plathelminthes); Flukes (Trematoda), e.g., Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp. and Nanocyctes spp, Cercomeromorpha, in particular Cestoda (Tapeworms), e.g., Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp, and Hymenolepis spp.

The present invention relates to the therapeutic and the non-therapeutic use of the mixtures of this invention and compositions comprising them for controlling and/or combating parasites in and/or on animals.

The mixtures of this invention and compositions comprising them may be used to protec t the animals from attack or infestation by parasites by contacting them with a parasitically effective amount of compounds of formula I. As such, “contacting” includes both direct contact (applying the mixtures/compositions directly on the parasite, including the application directly on the. animal or excluding the application directly on the animal, e.g., at it's locus for the latter) and indirect contact (applying the mixtures/compositions to the locus of the parasite), The contact of the parasite through application to its locus is an example of a non-therapeutic use of the mixtures of this invention and compositions comprising them.

“Locus” as defined above means the habitat, food supply, breeding ground, area, material or environment in which a parasite is growing or may grow outside of the animal. The mixtures of this invention and compositions comprising them can also be applied preventively to places at which occurrence of the posts or parasites is expected.

The mixtures of this invention and compositions comprising them can be effective through both contact (via soil, glass, wall, bed net, carpet, blankets or animal parts) and ingestion (e.g., baits).

The administration can be carried out prophylactically, therapeutically or non-therapeutically.

Administration of the active compounds used in the mixtures of this invention is carried out directly or in the form of suitable preparations, orally, topically/dermally or parenterally.

In general, “parasiticidally effective amount” means the amount of active compounds needed to achieve an observable effect on growth, including the effects of necrosis, death, retardation, prevention, and removal, destruction, or othenvise diminishing the occurrence and activity of the target organism. The parasiticidally effective amount can vary depending on the mixtures/compositions used in the inventtion. A parasiticidally effective amount of the mixtures/compositions will also vary accordina to the prevailing conditions such as desired parasitieidal effect and duration, target species, mode of application, and the like.

Generally it is favorable to apply the active compound(s) in total amounts of 0.5 mg/kg to 100 mg/kg per day, preferably 1 mg/kg to 50 mg/kg per day.

For oral administration to warm-blooded animals, the mixtures of this invention may be formulated as animal feeds, animal feed premixes, animal feed concentrates, pills, solutions, pastes, suspensions, drenches, gels, tablets, boluses and capsules. In addition, the mixtures of this invention may be administered to the animals in their drinkina water. For oral administration, the dosage form chosen should provide the animal with 0.01 mg/kg to 100 mg/kg or animal body weight per day of the active compound(s), preferably with 0.5 mg/kg to 100 mg/kg of animal body weight per day,

Alternatively, the mixtures of this invention may be administered to animals for example, by intraruminal, intramuscular, intravenous or subcutaneous injection. The formula I compounds may be dispersed or dissolved in a physiologically acceptable carrier for subcutaneous injection. Alternatively, the mixtures of this invention may be formulated into an implant for subcutaneous administration. In addition the mixtures of this invention may be transdermally administered to animals. For parenteral administration, the dosage form chosen should provide the animal with 0.01 mg/kg to 100 mg/kg of animal body weight per day of the mixtures of this invention.

The mixtures of this invention may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the mixtures of this invention. In addition, the mixtures of this invention may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection arc prepared by dissolving the active compounds in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g., dipropylenglycol monomethylether, ketones such as acetone, methylethylketone, aromatic hydrocarbons, vegetable and synthetic oils, dimethylformamide, dimethylacetamide, transcatol, propylencarbonate and mixtures thereof.

It may be advantageous to add thickeners during preparation. Suitable thickeners are inorganic thickeners such as bentonites, silicic acid, aluminum monostearate, organic thickeners such as cellulose derivatives, polyvinyl alcohols and their copolymers, acrylates and methacrylates.

Gels are applied to or spread on the Skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations arc poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropyene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, di-ethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-dioxolane and glycerol formal.

Suitable colorants are all colorants permitted for use on animals an which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections. Emulsions are either of the water-in-oil type or of the oil-in-water type.

They are prepared by dissolving the active compounds either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are: liquid paraffins, silicone oils, natural vegetable oils such as sesame oil, almond oil, castor oil, synthetic triglycerides such as caprylic/capric biglyceride, triglyceride mixture with vegetable fatty acids of the chain length C8-C12 or other specially selected natural fatty acids, partial glyceride mixtures of saturated or unsaturated fatty acids possibly also containing hydroxyl groups, mono- and diglycerides of the C8-C10 fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C16-C18, isopropyl myristate, isopropyl palmitate, caprylic/capric acid esters of saturated fatty alcohols or chain length C12-C18, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are: non-ionic surfactants, e.g., polyethoxylated castor oil, polyethoxylated sorbitan monooleate, sorbitan monostearatc, glycerol monostearate, polyoxyethyl stearate, alkylphenol polyglycol ether, ampholytic surfactants such as disodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolainine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures or the substances mentioned. Suspensions can be administered orally or topically/dermally. They are prepared by suspending the active compounds in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures. Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compounds are mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminum oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch crosslinked polyvinylpyrrolidone, binders such as starch, gelatin or linear polyvinylpyrrolidone, and dry binders such as microcrystalline cellulose.

The compositions which can be used in the invention can comprise generally from about 0.001 to 95% of the active compounds used in the mixture of this invention.

Ready-to-use preparations contain the mixtures of this invention acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the mixtures or this invention acting against ectoparasites in concentrations of 0.5 to 90 percent by weight, preferably of 1 to 50 percent by weight.

Furthermore, the preparations comprise the mixtures of this invention acting against endoparasites in concentrations of 10 ppm to 2 percent by weight, preferably of 0.05 to 0.9 percent by weight, very particularly preferably of 0.005 to 0.25 percent by weight.

The compositions comprising the mixtures of this invention can be applied orally, parenterally or topically, respectively dermally. For example, optionally the topical application is conducted in the form of compound-containing shaped articles such as collars, medallions, ear tags, bands for fixing at body parts, and adhesive strips and foils.

Generally it is favorable to apply solid formulations which release the active compounds of the mixtures of this invention in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the mixtures of this invention. A detailed list of plastics and elastomers as well as preparation procedures for the shaped articles is given e.g., in WO 03/086075.

The active compounds can also be used as a mixture with synergists or with other active compounds which act against pathogenic endo- and ectoparasites.

When applied in a mixture with synergists or with other active compounds the active compounds used in the mixture of this invention can be applied for example with synthetic coccidiosis compounds, polyetherantibiotics as Amprolium, Robenidin, Toltrazuril, Monensin, Salino-mycin, Maduramicin, Lasalocid, Narasin or Semduramicin.

Combinations of preferred embodiments with other preferred embodiments are within the scope of the present invention.

### EXAMPLES

### Biological Efficacy

Synergism can be described, as an interaction where the combined effect of two or more compounds is greater than the sum of the individual effects of each of the compounds. The presence of a synergistic effect in terms of percent control, between two mixing partners (X and Y) an be calculated using the Colby equation (Colby, S. R., 1967, Calculating Synergistic and Antagonistic Responses in Herbicide Combinations, Weeds, 15, 20-22);

$$E = X + Y - \frac{XY}{100}$$

When the observed combined control effect is greater than the expected combined control effect (E), then the combined effect is synergistic.

The following tests demonstrate the control efficacy of compounds, mixtures or compositions of this invention on specific pests. However, the pest control protection afforded by the compounds, mixtures or compositions is not limited to these species. In certain instances, combinations of a compound of this invention with other invertebrate pest control compounds or agents are found to exhibit synergistic effects against certain important invertebrate pests.

The analysis of synergism between the mixtures or compositions is determined using Colby's equation.

### Test B.1

For evaluating control of vetch aphid (Megoura viciae) through contact or systemic means the test unit consists of 24-well-microtiter plates containing broad bean leaf disks.

The compounds or mixtures are formulated using a solution containing 75 wt % water and 25 wt % DMSO. Different concentrations of formulated compounds or mixtures are sprayed onto the leaf disks at 2.5 μl, using a custom built micro atomizer, at two replications.

For experimental mixtures in these tests identical volumes of both mixing partners at the desired concentrations respectively, are mixed together.

After application, the leaf disks ore air-dried and 5-8 adult aphids are placed on the leaf disks inside the microtiter plate wells. The aphids are then allowed to suck on the treated leaf disks and incubated at 23+1° C., 50+5% RH (relative humidity) for 5 days. Aphid mortality and fecundity is then visually assessed.

### Test B.2.

For evaluating control of green peach aphid (Myzus persicae) through systemic means the test unit consists of 96-well-microtiter plates containing liquid artificial diet under an artificial membrane.

The compounds or mixtures are formulated using a solution containing 75 wt % water and 25 wt % DMSO. Different concentrations of formulated compounds or mixtures are pipetted into the aphid diet, using a custom built pipetter, at two replications.

For experimental mixtures in these tests identical volumes of both mixing partners at the desired concentrations respectively, are mixed together.

After application, 5-8 adult aphids are placed on the artificial membrane inside the microtiter plate wells. The aphids are then allowed to suck on the treated aphid diet and are incubated at 23+1° C., 50+5% RH for 3 days. Aphid mortality and fecundity is then visually assessed.

### Test B.3

For evaluating control of boll weevil (Anthonomus grandis) the test unit consists of 24-well-microtiter plates containing an insect diet and 20-30 A. grandis eggs.

The compounds or mixtures are formulated using a solution containing 75 wt % water and 25 wt % DMSO. Different concentrations of formulated compounds or mixtures are sprayed onto the insect diet at 20 μl, using a custom built micro atomizer, at two replications.

For experimental mixtures in these tests identical volumes of both mixing partners at the desired concentrations respectively, are mixed together.

After application, microtiter plates are incubated at 23±1° C., 50±5% for 5 days. Egg and larval mortality is visually assessed.

### Test B.4

For evaluating control of Mediterranean fruitfly (Ceratitis capitata) the test unit consists of 96-well-microtiter plates containing an insect diet and 50-80 C, capitata eggs.

The compounds or mixtures are formulated using a solution containing 75 wt % water and 25 wt % DMSO. Different concentrations of formulated compounds or mixtures are sprayed onto the insect diet at 5 μl, using a custom built micro atomizer, at two replications.

For experimental mixtures in these tests identical volumes of both mixing partners at the desired concentrations respectively, are mixed together.

After application, microtiter plates are incubated at 28±1° C., 5% RH for 5 days. Egg and larval mortality is visually assessed.

### Test B.5

For evaluating control of tobacco budworm (Heliothis virescens) the test unit consists of 96-well-microtiter plates containing an insect diet and 15-25 H. virescens eggs.

The compounds or mixtures are formulated using a solution containing 75 wt % water and 25 wt % DMSO. Different concentrations of formulated compounds or mixtures are sprayed onto the insect diet at 10 μl, using a custom built micro atomizer, at two replications.

For experimental mixtures in these tests identical volumes of both mixing partners at the desired concentrations respectively, are mixed together.

After application, microliter plates are incubated at 28±1° C., 80±5% RH for 5 days. Egg and larval mortality is visually assessed.

### Test B.6

For evaluating control of bird cherry aphid (Rhopalosiphum padi) through contact or systemic means the test unit consists of 96-well-microtiter plates containing barley leaf disks.

The compounds or mixtures are formulated using a solution containing 75 wt % water and 25 wt % DMSO. Different concentrations of formulated compounds or mixtures are sprayed onto the leaf disks at 2.5 μl, using a custom built micro atomizer, at two replications.

For experimental mixtures in these tests identical volumes of both mixing partners at the desired concentrations respectively, are mixed together.

After application, the leaf disks are air-dried and 5-8 adult aphids placed on the leaf disks inside the microtiter plate wells. The aphids are then allowed to suck on the treated leaf disks and incubated at 25+1° C., 80+5% RH for 3 to 5 days. Aphid mortality and fecundity is visually assessed.

### Test B.7

For evaluating control of southern armyworm (Spodoptera eridania) the test unit consists of Petri dishes containing eight-day-old Henderson bush lima bean leaves and 10 neonate S. eridania larvae.

The compounds or mixtures are formulated using a solution containing 99.05 vol % water and 0.05 vol % of a surfactant (Kinetic). Leaves are dipped into different concentrations of formulated compounds or mixtures and allowed to dry. Four replications are performed.

For experimental mixtures in these tests identical volumes of both mixing partners at the desired concentrations respectively, are mixed together.

After application, Petri dishes are incubated in the dark at 26±1° C., for 4 days. Larval mortality is visually assessed.

Metaflumizone at rates of 2.5 and 5.0 kg ai/ha and Streptomyces galbus strain M1064 at rates of 0.69. 1.39, and 2.79 kg ai/ha were tested singly and in combination for control of southern armyworm, Spodoptera eridania in the laboratory. The results of these tests are summarized in the following Tables 1 and 2:

TABLE 1

| | Application Rate | Observed Mortality |
|---|---|---|
| Active Ingredient | [kg ai/ha] | [%] |
| Streptomyces galbus strain | 0.69 | 7.5 |
| M1064 | 1.39 | 20.0 |
| | 2.79 | 92.5 |
| Metaflumizone | 2.5 | 15.0 |
| | 5.0 | 47.5 |

TABLE 2

| | Application | Observed | Expected |
|---|---|---|---|
| | Rates | Mortality | Mortality |
| Active Ingredients | [kg ai/ha] | [%] | [%] |
| Streptomyces galbus strain | 0.69 | 80.0 | 22.5 |
| M1064 + Metaflumizone | +2.5 | | |
| Streptomyces galbus strain | 1.39 | 70.0 | 35.0 |
| M1064 + Metaflumizone | +2.5 | | |
| Streptomyces galbus strain | 0.69 | 75.0 | 55.0 |
| M1064 + Metaflumizone | +5 | | |
| Streptomyces galbus strain | 1.39 | 100.0 | 67.5 |
| M1064 + Metaflumizone | +5 | | |

The results indicate synergy according to the Colby equation with combinations of Streptomyces galbus M1064 and metaflumizone.

1. A pesticidal mixture comprising, as active compounds,
1) at least one active compound I selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and
2) at least one active compound II selected from the groups A.1 to A.27:
A.1. Organo(thio)phosphate compounds selected from the group consisting of acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphosmethyl, profenofos, propetamphos, prothiofos, pyraclofos, pyridaphenthion, quinalphos, sulfotep, tebupirimfos, temephos, terbufos, tetra-chlorvinphos, thiometon, triazophos, trichlorfon and vamidothion;
A.2. Carbamate compounds selected from the group consisting of aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate;
A.3. Pyrethroid compounds selected from the group consisting of acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, pro-fluthrin, pyrethrin (pyrethrum), resmethrin, silafluofen, tefluthrin, tetramethrin, tralomethrin and transfluthrin;
A.4. Juvenile hormone mimics selected from the group consisting of hydroprene, kinoprene, methoprene, fenoxycarb and pyriproxyfen;
A.5. Nicotinic receptor agonists/antagonists compounds selected from the group consisting of acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.
A.6. GABA gated chloride channel antagonist compounds selected from the group consisting of chlordane, endosulfan, gamma-HCH (lindane); ethiprole, fipronil, pyrafluprole and pyriprole
A.7. Chloride channel activators selected from the group consisting of abamectin, emamectin benzoate, milbemectin and lepimectin;
A.8. METI I compounds selected from the group consisting of fenazaquin, fenpyroximate, pyrimidifen, pyridaben, tebufenpyrad, tolfenpyrad, flufenerim and rotenone;
A.9. METI II and III compounds selected from the group consisting of acequinocyl, fluacyprim and hydramethylnon;
A.10. Uncouplers of oxidative phosphorylation selected from the group consisting of chlorfenapyr and DNOC;
A.11. Inhibitors of oxidative phosphorylation selected from the group consisting of azocyclotin, cyhexatin, diafenthiuron, fenbutatin oxide, propargite and tetradifon;
A.12. Moulting disruptors selected from the group consisting of cyromazine, chromafenozide, halofenozide, methoxyfenozide and tebufenozide;
A.13. Synergists selected from the group consisting of piperonyl butoxide and tribufos;
A.14. Sodium channel blocker compounds selected from the group consisting of indoxacarb and metaflumizone;
A.15. Fumigants selected from the group consisting of methyl bromide, chloropicrin and sulfuryl fluoride;
A.16. Selective feeding blockers selected from the group consisting of crylotie, pymetrozine and flonicamid;
A.17. Mite growth inhibitors selected from the group consisting of clofentezine, hexythiazox and etoxazole;
A.18. Chitin synthesis inhibitors selected from the group consisting of buprofezin, bistrifluron, chlorfluazuron, diflubenzuron, flucycloxuron, flufenoxuron, hexaflumuron, lufenuron, novaluron, noviflumuron, teflubenzuron and triflumuron;
A.19. Lipid biosynthesis inhibitors selected from the group consisting of spirodiclofen, spiromesifen, and spirotetramat;
A.20. Octapaminergic agonsits: amitraz;
A.21. Ryanodine receptor modulators: flubendiamide and the phtalamid compound (R)-, (S)-3-Chlor-N1-{2-methyl-4-[1,2,2,2-tetrafluor-1-(trifluormethyl)ethyl]phenyl }-N2-(1-methyl-2-methylsulfonylethyl)phthalamid (A.21.1)
A.22. soxazoline compounds selected from the group consisting of 4-[5-(3,5-Dichloro-phenyl)-5-trifluoromethyl-4,5-dihydro-isoxazol-3-yl]-2-methyl-N-pyridin-2-ylmethyl-benzamide (A.22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (A.22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (A.22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]amide (A.22.4) and 4-[5-(3,5-Dichlorophenyl)-5-trifluoromethyl-4,5-dihydro-isoxazol-3-yl]-N-[(methoxyimino)methyl]-2-methylbenzamide (A.22.5);
A.23. Anthranilamide compounds selected from the group consisting of chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (A.23.1), 5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (A.23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (A.23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (A.23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (A.23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (A.23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (A.23.7), N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (A.23.8), N′-(2-{[5-Bromo-2-(3-chloro-pyridin-2-yl)-2H-pyrazole-3-carbonyl]-amino}-5-chloro-3-methylbenzoyl)-N,N′-dimethyl-hydrazinecarboxylic acid methyl ester (A.23.9), N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (A.23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (A.23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (A.23.12);
A.24. Malononitrile compounds selected from the group consisting of 2-(2,2,3,3,4,4,5,5-octafluoropentyl)-2-(3,3,3-trifluoro-propyl)malononitrile (CF2H—CF2-CF2-CF2-CH2-C(CN)2-CH2-CH2-CF3) (A.24.1) and 2-(2,2,3,3,4,4,5,5-octafluoropentyl)-2-(3,3,4,4,4-pentafluorobutyl)-malonodinitrile (CF2H—CF2-CF2-CF2-CH2C(CN)2-CH2-CH2-CF2-CF3) (A.24.2);
A.25. Microbial disruptors selected from the group consisting of Bacillus thuringiensis subsp. Israelensi, Bacillus sphaericus, Bacillus thuringiensis subsp. Aizawai, Bacillus thuringiensis subsp. Kurstaki, and Bacillus thuringiensis subsp. Tenebrionis;
A.26. Aminofuranone compounds selected from the group consisting of 4-{[(6-Bromopyrid-3-yl)methyl](2-fluoroethyl)amino}furan-2(5H)-on (A.26.1), 4-{[(6-Fluoropyrid-3-yl)methyl](2,2-difluoroethyl)amino}furan-2(5H)-on (A.26.2),4-{[(2-Chloro1,3-thiazolo-5-yl)methyl](2-fluoroethyl)amino}furan-2(5H)-on (A.26.3), 4-{[(6-Chloropyrid-3-yl)methyl](2-fluoroethyl)amino}furan-2(5H)-on (A.26.4), 4-{[(6-Chloropyrid-3-yl)methyl](2,2-difluoroethyl)amino}furan-2(5H)-on (A.26.5), 4-{[(6-Chloro-5-fluoropyrid-3-yl)methyl](methyl)amino}furan-2(5H)-on (A.26.6), 4-{[(5,6-Dichloropyrid-3-yl)methyl](2-fluoroethyl)amino}furan-2(5H)-on (A.26.7), 4-{[(6-Chloro-5-fluoropyrid-3-yl)methyl](cyclopropyl)amino}furan-2(5H)-on (A26.8), 4-{[(6-Chloropyrid-3-yl)methyl](cyclopropyl)amino}furan-2(5H)-on (A.26.9) and 4-{[(6-Chloropyrid-3-yl)methyl](methyl)amino}furan-2(5H)-on (A.26.10);
A.27. Various compounds selected from the group consisting of aluminum phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N—R′-2,2-dihalo-1-R″cyclopropanecarboxamide-2-(2,6-dichloro-α, α, α-trifluoro-p-tolyl)hydrazone or N—R′-2,2-di(R′″)propionamide-2-(2,6-dichloro-α, α, α-trifluoro-p-tolyl)-hydrazone, wherein R′ is methyl or ethyl, halo is chloro or bromo, R″ is hydrogen or methyl and R′″ is methyl or ethyl, 4-But-2-ynyloxy-6-(3,5-dimethylpiperidin-1-yl)-2-fluoro-pyrimidine (A.27.1), Cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester (A.27.2) and 8-(2-Cyclopropylmethoxy-4-trifluoromethyl-phenoxy)-3-(6-trifluoromethylpyridazin-3-yl)-3-aza-bicyclo[3.2.1]octane (A.27.3) in synergistically effective amounts.

2. The mixture according to claim 1, wherein the active compound I is the Streptomyces galbus strain having accession number NRRL 30232 or the Streptomyces galbus strain having accession number NRRL 50334.

3. The mixture according to claim 1, wherein the active compound II is selected from the groups A.1, A.2, A.3, A.5, A.6, A.7, A.10, A.12, A.14, A.18, A.20, A.21, A.22, A.23, A.25, and A.27.

4. The mixture according to claim 3, wherein the active compound II is selected from the group consisting of acephate, chlorpyrifos, diazinon, carbaryl, methomyl, allethrin, bifenthrin, cyfluthrin, lambda-cyhalothrin, cypermethrin, alpha-cypermethrin, beta-cypermethrin, zeta-cypermethrin, deltamethrin, etofenprox, fenpropathrin, fenvalerate, flucythrinate, pyrethrin, taufluvalinate, silafluofen, tralomethrin, thiamethoxam, spinosad, fipronil, emamectin benzoate, lepimectin, halofenozide, chlorfenapyr, indoxacarb, metaflumizone, lufenuron, novaluron, amitraz, flubendiamide, 4-[5-(3,5-Dichloro-phenyl)-5-trifluoromethyl-4,5-dihydro-isoxazol-3-yl]-2-methyl-N-pyridin-2-ylmethyl-benzamide, chloranthraniliprole, cyantraniliprole, Bacillus thuringiensis subsp. Kurstaki, and pyridalyl.

5. The mixture according to claim 4, wherein the active compound II is metaflumizone.

6. The mixture according to claim 1, comprising the active compound I and the active compound II in a weight ratio of from 600:1 to 1:100.

7. A composition, comprising a mixture as defined in claim 1 and at least one liquid or solid carrier.

8. A method for protecting growing plants from attack or infestation by insects, acarids or nematodes comprising contacting the plant, or the soil or water in which the plant is growing, with a pesticidal mixture comprising at least one active compound I selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and an active compound II selected from the group consisting of acephate, chlorpyrifos, diazinon, carbaryl, methomyl, allethrin, bifenthrin, cyfluthrin, lambda-cyhalothrin, cypermethrin, alpha-cypermethrin, beta-cypermethrin, zeta-cypermethrin, deltamethrin, etofenprox, fenpropathrin, fenvalerate, flucythrinate, pyrethrin, taufluvalinate, silafluofen, tralomethrin, thiamethoxam, spinosad, fipronil, emamectin benzoate, lepimectin, halofenozide, chlorfenapyr, indoxacarb, metaflumizone, lufenuron, novaluron, amitraz, flubendiamide, 4-[5-(3,5-Dichloro-phenyl)-5-trifluoromethyl-4,5-dihydro-isoxazol-3-yl]-2-methyl-N-pyridin-2-ylmethyl-benzamide, chloranthraniliprole, cyantraniliprole, Bacillus thuringiensis subsp. Kurstaki, and pyridalyl in synergistically effective amounts.

9. A method for combating or controlling insects, acarids or nematodes comprising contacting an insect, acarid or nematode or their food supply, habitat, breeding grounds or their locus with a pesticidal mixture comprising at least one active compound I selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and an active compound II selected from the group consisting of acephate, chlorpyrifos, diazinon, carbaryl, methomyl, allethrin, bifenthrin, cyfluthrin, lambda-cyhalothrin, cypermethrin, alpha-cypermethrin, beta-cypermethrin, zeta-cypermethrin, deltamethrin, etofenprox, fenpropathrin, fenvalerate, flucythrinate, pyrethrin, taufluvalinate, silafluofen, tralomethrin, thiamethoxam, spinosad, fipronil, emamectin benzoate, lepimectin, halofenozide, chlorfenapyr, indoxacarb, metaflumizone, lufenuron, novaluron, amitraz, flubendiamide, 4-[5-(3,5-Dichloro-phenyl)-5-trifluoromethyl-4,5-dihydroisoxazol-3-yl]-2-methyl-N-pyridin-2-ylmethyl-benzamide, chloranthraniliprole, cyantraniliprole, Bacillus thuringiensis subsp. Kurstaki, and pyridalyl in synergistically effective amounts.

10. A method for the protection of plant propagation material from pests comprising contacting the plant propagation material with a pesticidal mixture comprising at least one active compound I selected from the group consisting of the Streptomyces galbus strain having accession number NRRL 30232, the Streptomyces galbus strain having accession number NRRL 50334, a mutant of said strains, a variant of said strains, a metabolite produced by said strains, a supernatant obtained from the whole broth culture of said strains and a solvent extract of said supernatants, wherein said mutant and variant have the identifying characteristics substantially identical to those of said strains, and an active compound II selected from the group consisting of acephate, chlorpyrifos, diazinon, carbaryl, methomyl, allethrin, bifenthrin, cyfluthrin, lambda-cyhalothrin, cypermethrin, alpha-cypermethrin, beta-cypermethrin, zeta-cypermethrin, deltamethrin, etofenprox, fenpropathrin, fenvalerate, flucythrinate, pyrethrin, taufluvalinate, silafluofen, tralomethrin, thiamethoxam, spinosad, fipronil, emamectin benzoate, lepimectin, halofenozide, chlorfenapyr, indoxacarb, metaflumizone, lufenuron, novaluron, amitraz, flubendiamide, 4-[5-(3,5-Dichloro-phenyl)-5-trifluoromethyl-4,5-dihydro-isoxazol-3-yl]-2-methyl-N-pyridin-2-ylmethyl-benzamide, chloranthraniliprole, cyantraniliprole, Bacillus thuringiensis subsp. Kurstaki, and pyridalyl in synergistically effective amounts.

11. (canceled)

12. (canceled)

13. The method according to claim 8 wherein the active compound I and the active compound II are applied simultaneously, that is jointly or separately, or in succession.

14. (canceled)

15. (canceled)
